(19) United States
(12) Patent Application Publication
(10) Pub. No.: US 2007/0173786 A1
(43) Pub. Date: Jul. 26, 2007

(54) FLUID INJECTION SYSTEM AND METHOD USING MULTI-LUMEN CATHETERS

(75) Inventors: DANIEL K. RECINELLA, Queensbury, NY (US); Katherine C. McCarty, Hudson Falls, NY (US)

Correspondence Address: Harry K. Ahn, Esq.;Reed Smith LLP, 599 Lexington Avenue, New York, NY, 10022, US

(73) Assignee: AngioDynamics, Inc.

(21) Appl. No.: 11/627,308

(22) Filed: Jan. 25, 2007

Related U.S. Application Data
(60) Provisional application No. 60/762,358, filed on Jan. 25, 2006.

(51) Int. Cl. A61M 25/00

(52) U.S. Cl. 604/523

(57) ABSTRACT

A fluid injection device including a multi-lumen catheter and a coupler is provided. The catheter has a common tube portion containing a set of lumens and a distal end adapted to be placed in a blood vessel. At least two extension tubes extend from the common tube portion. Output connectors of the coupler are coupled to the catheter's extension tubes such that they are combined into an input lumen which is coupled to a fluid source to simultaneously inject the fluid into the catheter's extension tubes. The simultaneous injection of the fluid into the multi-lumen catheter's extension tubes provides an even pressure on the lumen walls of the catheter, a higher flow rate and a balanced thrust at the exit of the catheter.

### CROSS-REFERENCE TO RELATED APPLICATIONS

This application claims priority to U.S. Provisional Patent Application Ser. No. 60/762,358, filed Jan. 25, 2006, which is incorporated herein by reference.

### TECHNICAL FIELD

The present invention relates to a medical device and method, and more particularly, a device and method for injecting fluid into a blood vessel.

### BACKGROUND OF THE INVENTION

A common method for performing diagnostic imaging of blood vessels is to inject into a blood vessel a dye, or contrast media, that is visible under fluoroscopic or computer tomographic (CT) imaging. CT imaging takes a rapid stream of X-ray photographs from different angles. Through computerization, this block of data is used to create two- and three-dimensional images of bone and other hard tissue, and soft tissue when contrast media is introduced inside the body.

CT injectors are used in CT imaging to inject a controlled volume of contrast media or dye at a high pressure into catheters that are inserted into a blood vessel. Some advantages of intravenously delivering contrast media by means of power injection for CT include uniform contrast-medium delivery, optimal timing of delivery, and decreased radiation and needle exposure to health-care providers. Before the contrast media or dye is injected, the catheter is typically flushed with a solution such as saline to ensure that the catheter is not occluded or damaged. When the dye is injected it mixes with the blood, makes the blood less permeable by x-ray, and allows the vasculature to be visualized. This also allows direct access to the flowing blood. The images that are obtained show the outline of the blood vessel where the dye flows.

Commonly, needles are placed in a vein for CT contrast injection. These needles are generally placed in the arm or other peripheral vein. The contrast media or dye travels through the venous system to the heart where it is distributed throughout the body. CT injections are usually done at rates ranging from 1-10 cc/sec and deliver 50-200 cc in total volume. During CT injections, the small peripheral veins may be subject to undue stress where the needle is placed. The needle access can destroy the veins and can cause further serious complications such as extravasation, an accidental infiltration of contrast media into surrounding tissue.

On many occasions the patients needing CT imaging are very ill. Commonly, these patients will have a peripherally inserted central catheter (PICC) or central venous catheter (CVC) in place for the delivery of therapeutic solutions to assist in their treatment. These catheters can have single or multiple lumens. PICCs typically provide short or long term peripheral access to the central venous system for intravenous therapy, blood sampling and CT injection. A PICC is inserted in a peripheral vein, such as the cephalic vein, basilic vein, or brachial vein and then advanced through increasingly larger veins, toward the heart until the tip rests in the distal superior vena cava or cavo-atrial junction. In comparison, a central venous catheter (CVC) is placed into a large vein such as the internal jugular vein, the subclavian vein, or the femoral vein.

Typically, these catheters are made from a soft polymer material and are capable of withstanding the pressure from a slow infusion pump or gravity feed from a hanging bag. These pumps or bags generate low pressures and do not put excessive stress on the external walls of the catheter or, in the case of a multi-lumen catheter, on the septum between the lumens.

It is very common to have a need for obtaining images from patients that have various medical conditions (cancer, infections, etc.). The typical method for obtaining images, as described above, involves inserting a needle into a peripheral vein and injecting dye. Placing a needle into a vein can be traumatic and painful for patients, as it puts the patient through another invasive medical procedure and also can destroy a vein that may be needed in the future for other medical interventions. It can also be time-consuming for medical personnel.

Another method for performing CT imaging with these patients is to perform a CT injection through an existing PICC that is already in place inside of a blood vessel, avoiding the need for an additional access site. The ability to inject a dye through the catheter that is already in place saves time, money, and the patient from another medical procedure to place a catheter or needle only for dye injections. Further, contrast injections through PICCs' deliver contrast to the central circulation thereby providing better mixing and better images with less total contrast delivered. Additionally, there are no shearing forces from the injection being applied to the walls of small veins. For these reasons it is desirable to use a central catheter vs. a peripheral needle for the patient's benefit.

The current method for injecting contrast dye through PICCs' is inadequate. The output of the CT injector has a single connector for attaching to the catheter. This allows the injection to go through only one of the catheter lumens. CT injectors can generate pressures up to 300 psi. The full force of the injection is seen by only one lumen of the catheter. This limits the flow rate that can be generated which can result in an unacceptable imaging procedure. The catheter can be pushed to its limit trying to obtain high flow rates by increasing the delivery pressure. This could cause the catheter to burst, which will result in injury to the patient. In either case, an imaging study would need to be repeated, putting the patient through another medical procedure.

The quality of the images obtained with the injected dye is partially dependent on the flow rates delivered. In many cases the lumens of catheters with multiple lumens can be small in size. These small caliber lumens can make it difficult to obtain the dye injection flow rates needed for optimal images. Although CT injectable catheters have been developed in response to this problem, many of these catheters have only one lumen. Injections through one lumen of a multi-lumen catheter can stress the internal wall or septum of the catheter with the great pressure difference between the different lumens. The injections must also be done at a high bolus and a high concentration in order to be effective. Moreover, injections through a single lumen of a multi-lumen catheter create an eccentric thrust at the exit of the catheter that can cause the catheter to move into an unwanted position.

Further, as the imaging technology becomes more advanced, the flow rate of contrast dye required to obtain the images is increasing. Older CT technology requires 1-5 cc/sec of contrast media. This is achievable with a needle and with injecting through a single lumen of a multi-lumen PICC catheter. However, flow rates such as these can lead to inadequate contrast enhancement of organs. Newer technology requires a minimum of 6 cc/sec contrast flow rate. Therefore, it is desirable to provide an improved injection device and method for CT injection which provides such higher flow rate and reduces or eliminates the problems associated with internal wall stress and eccentric thrust.

Without limiting the scope of the invention, a brief summary of the disclosure is set forth below. Additional details of the disclosure and/or additional embodiments of the invention may be found in the Detailed Description.

### BRIEF SUMMARY OF THE DISCLOSURE

The present invention addresses the need for an effective way to safely and more effectively increase flow rates through a multi-lumen catheter by using all lumens of the catheter.

In one aspect, a fluid injection device including a multi-lumen catheter and a coupler is provided. The multi-lumen catheter has a common tube portion containing a set of lumens and a distal end adapted to be placed in a blood vessel. At least two extension tubes extend from the common tube with each having a lumen in communication with a respective one of the lumens in the common tube portion. Output connectors of the coupler are coupled to the respective lumens of the catheter's extension tubes such that they are combined into an input lumen. The input lumen of the coupler is adapted to be connected to a fluid source to simultaneously inject the fluid into the lumens of the catheter's extension tubes.

In another aspect, the coupler is a multi-lumen extension line having its extension lines connected to the respective lumens of the catheter's extension tubes.

In another aspect, a method of injecting fluid through a multi-lumen catheter is provided. The extension tubes of the multi-lumen catheter are connected to a source of the fluid. Then the fluid is simultaneously injected into the lumens of the extension tubes.

The simultaneous injection of the fluid into at least two lumens of the multi-lumen catheter results in an even pressure on the lumen walls/septum of the catheter, thereby providing an effective way to increase flow rates, decrease the incidence of eccentric flow, and provide a balanced thrust at the exit of the catheter.

These and various other objects, advantages and features of the invention will become apparent from the following description and claims, when considered in conjunction with the appended drawings. The invention will be explained in greater detail below with reference to the attached drawings. Several embodiments of the present invention are described below, in which the same reference characters denote similar elements.

### BRIEF DESCRIPTION OF THE DRAWINGS

FIG. 1A illustrates a plan view of a prior art method of injecting dye through a catheter.

FIG. 1B illustrates a plan view of the system for injecting dye through a dual lumen catheter in accordance with the method of the current invention.

FIG. 1C illustrates a plan view of the system for injecting dye through a triple lumen catheter in accordance with the method of the current invention.

FIG. 2A illustrates a cross-sectional view of a dual and triple lumen catheter showing the effect of pressure when one lumen is used for the injection.

FIG. 2B illustrates a cross-sectional view of a dual and triple lumen catheter showing the effect of pressure when all lumens are used for the injection.

FIG. 3A illustrates a partial side view of the distal end of the catheter showing movement of the distal end of the catheter when only one lumen is used for injection.

FIG. 3B illustrates a partial side view of the distal end of the catheter showing the distal end of the catheter when the method of the current invention is used for injection.

### DETAILED DESCRIPTION OF THE INVENTION

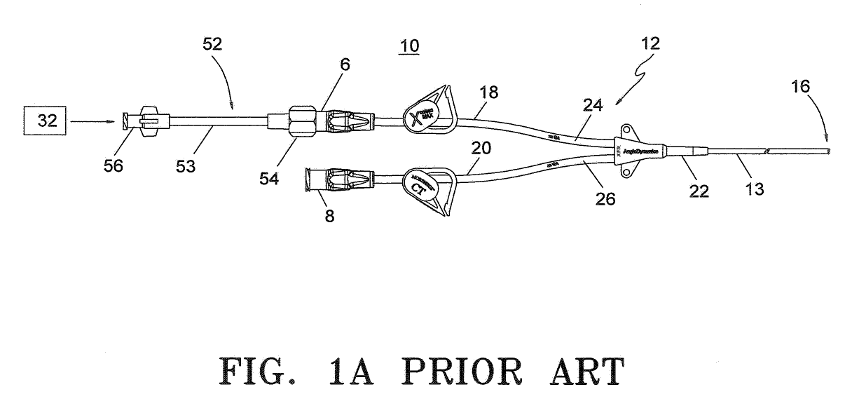

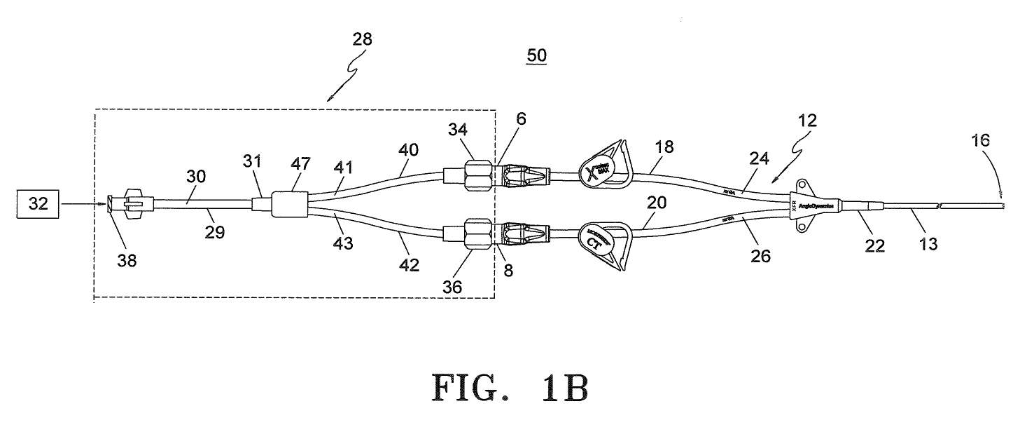

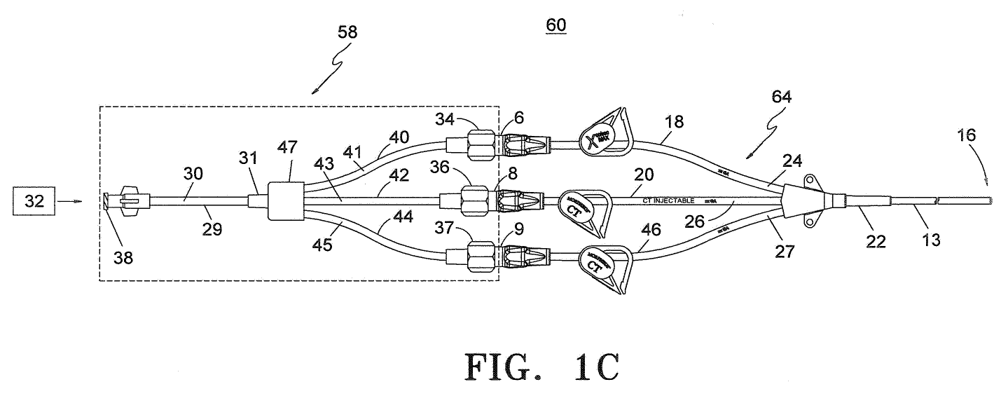

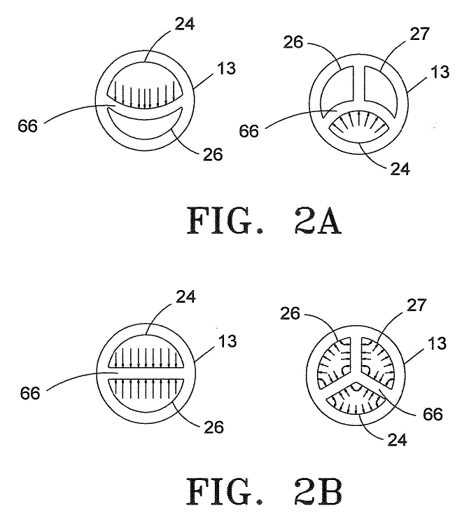

FIG. 1A illustrates a prior art injection device 10 which includes a dual-lumen catheter 12 and an extension piece 52. The catheter 12 has a common tube portion 13 having a set of lumens 24, 26 (see FIG. 2A also) and a distal end 16 which is adapted to be placed in a blood vessel of a patient. A pair of extension tubes 18, 20 extend from a proximal end 22 of the common tube 13 with each extension tube 18, 20 having a single lumen in communication with a respective one of the lumens 24, 26. The proximal end of each extension tube 18, 20 includes a Luer-type connector 6, 8 for connection to the extension piece 52.

The extension piece 52 includes a common extension line 53 having a through lumen, a distal connector 54 connected to the extension tube 18 of the catheter through the connector 6 and a proximal connector 56 for connection to a fluid source 32.

In such a conventional injection device 10, only one lumen of the multi-lumen catheter 12 is used to inject the contrast media. One disadvantage of such a conventional method is that the amount of fluid flow is limited due to the size of the lumen. In many instances the maximum flow rate that can be obtained is not adequate. Another disadvantage of the conventional injection method is that it creates unequal pressures on the lumen walls, which causes distension of the inner septum that can lead to catheter weakening or failure. Still another disadvantage of injecting through a single lumen is that the injection results in an eccentric thrust at the distal tip/end of the multi-lumen catheter. This can cause catheter tip movement or whipping. Eccentric thrust is defined as an impulsive fluid force with a line of action which does not pass through the center of gravity of a body, such as a catheter, thereby causing angular acceleration which may result in a bending moment of the body.

By contrast, the injection device according to the present invention involves simultaneously using multiple lumens, rather than a single lumen, of the multi-lumen catheter. One embodiment of the present invention is shown in FIG. 1B in which a multi-lumen PICC 12 has been inserted into a patient's blood vessel.

The multi-lumen catheter of the present invention may be connected to a coupler device 28/58 capable of receiving multiple lumens, as illustrated in FIGS. 1B and 1C. Instead of the extension piece 52, which connects to only a single lumen of the multi-lumen catheter 12, a fluid injection device 50 of FIG. 1B includes a coupler device 28 which provides a fluid communication pathway between the fluid source 32 and the lumens 24, 26 of the extension tubes 18, 20 of the catheter 12. In relation to the fluid source 32, the coupler device 28 can also be viewed as a splitter device that splits the common extension line portion 29 into two separate extension lines 40 and 42, which are in fluid communication with extension tubes 18, 20.

In FIG. 1B, the coupler device 28 is a multi-lumen extension line that includes a common line portion 29 having a single lumen 30 and a proximal connector 38 for connection to the fluid source 32, such as a contrast media injector. A pair of extension lines 40, 42 extend from a distal end 31 of the common line 29 with each extension line 40,42 having a distal connector 34, 36 that connects to the proximal connectors 6, 8 of the catheter 12. Thus, the lumen 41 of the extension line 40 is in communication with the lumen 24 of the catheter extension tube 18, while the lumen 43 of the extension line 42 is in communication with the lumen 26 of the catheter extension tube 20.

The coupler device 28 is connected to the multi-lumen catheter 12 so that the lumens 18, 20 of the catheter 12 are combined into a single lumen 30 by the coupler device 28. A fluid source 32 is connected to the coupler device 28 through the proximal connector 38. The fluid source 32 is then turned on so that the fluid is simultaneously injected into the two lumens 24, 26 of the multi-lumen catheter 12.

FIG. 1C shows an alternative injection device 60. In this embodiment, the coupler/splitter device 58 is designed for a triple lumen catheter 64 similar to the two-lumen catheter 12 of FIG. 1B. The lumen 30 splits into three lumens 41, 43, 45 respectively contained in three extension lines 40, 42, 44. These lines, in turn, are respectively in communication with three lumens 24, 26, 27 of the catheter extension tubes 18, 20, 46. The distal connectors 34, 36, 37 of the coupler device 58 are connected to proximal connectors 6, 8, 9 of the triple-lumen catheter 64. As can be seen, the design of the present invention can be expanded to a multi-lumen catheter of any number of lumens. The catheter of the present invention may be composed of any suitable plastic material, such as but not limited to, urethane, nylon, or silicone. The catheter may be of any useable length, i.e., from the proximal end of the catheter 22 to the distal end of the catheter 16, up to about 65 cm.

Such a device provides an increased flow rate of the fluid by delivering the fluid into all lumens of the catheter 12 simultaneously and proportionately. This can be accomplished by using a coupler 28/58 that connects the extension lines 40, 42 with the extension tubes 18, 20, thereby providing a single continuous fluid pathway. This expands the overall flow area of the catheter 12 by the cross sectional area of the added lumen or lumens. This also increases the amount of fluid that can be delivered as is described by the controlling equation, known as Poiseuille's Law, which states that:

$$Q = \frac{π \cdot Δ P \cdot {D}^{4}}{128 \cdot μ \cdot L}$$

### Where π=a constant of 3.1415926;

ΔP Pressure gradient (psi);

D=Hydraulic Diameter (meters);

μ=viscosity of fluid (Pa*s); and

L=length of the flow path (meters).

The hydraulic diameter D equals 4*A/P, where A=the cross sectional area of the catheter lumen, and P equals the perimeter of the lumen. The rate of flow through a rigid tube (Q) is equal to the fourth power of the diameter and the difference in pressure at the two ends of the tube and is inversely related to the viscosity of the fluid and the length of the tube. Although this equation is accurate only for laminar flow in a rigid tube, this is a reasonable approximation for fluid flow through a catheter tube, as in the device and method of the present invention. The elasticity of the PICC used in the embodiments of the present invention will result in somewhat lower actual pressures.

By way of example, in the above equation, if all other parameters stay the same, an increase in the diameter of the catheter will cause the flow rate to increase. For higher viscosity fluids, increasing the diameter of the flow area will cause the flow rate to remain the same. Table 1 below illustrates the effect on the flow rate of different hydraulic diameters. For example, as the cross-sectional area of the catheter increases, the flow rate increases.

TABLE 1

| | |
|---|---|
| Result | Inputs |

| Flow rate | Combined Catheter Area | Perimeter |
|---|---|---|
| ml/s | in² | in |
| 6.02 | 0.0008 | 0.1101 |
| 6.32 | 0.001 | 0.136 |
| 6.43 | 0.0012 | 0.1626 |
| 6.49 | 0.0014 | 0.1891 |

Any suitable therapeutic fluid may be injected from the fluid source 32 into the catheter 12 using the method of the present invention. Such therapeutic fluids include, but are not limited to, contrast media or dye, chemotherapeutic agents or drugs, anti-thrombotic agents, parenteral nutritional fluids, antibiotic and antiviral fluids, IV fluids, and others. These fluids may be of differing viscosities and may be injected simultaneously through different lumens of the catheter. Contrast agents such as contrast media or dye may be combined with these other agents, before being injected into the catheter 12, to provide additional clinical advantages, or alternatively, the contrast agent may be injected separately from any additional fluids. The contrast agents will mix within the blood flow once the fluid has exited from the distal end of the catheter 16, thereby providing an enhanced contrast image.

Any suitable contrast media may be used. Examples of contrast agents that may be used may include, but are not limited to, gadolinium, Isovue®-370, Hexabrix-320, Isovist-240, Optiray-320, Omnipaque-350, and Ultravist-370, 43% meglumine iothalamate, lohexol 240, and 60% meglumine iothalamate. A typical contrast media has a viscosity in the range of 0.07-0.11 poise. By way of example, if a contrast media with a viscosity of 0.10 poise were to be injected through a catheter with a 60 cm length, a flow hydraulic diameter of 0.0118 inches, and an injection pressure of 250 psi, the flow rate would be 5.6 ml/sec. If the contrast media is injected through two lumens at the same time while doubling the hydraulic diameter and keeping all other variables constant, the flow rate will increase to 11.2 ml/sec.

Alternatively, the hydraulic diameter of the catheter can be doubled by using two catheter lumens, and the flow rate can be kept the same by decreasing the amount of pressure needed to obtain the flow rate. For example, using the equation above, to obtain 5.2 ml/sec with a doubled diameter, a pressure of 125 psi would be needed, rather than 250 psi, to obtain the necessary flow rate. Low pressure injections provide additional clinical advantages of decreased incidence of catheter weakening or failure and less potential trauma to the vein of the patient. Yet another alternative to increase the flow rate would be to maintain the pressure and increase the viscosity. Using fluids of higher viscosities provides an advantage in CT injections because the higher viscosity fluids provide higher contrast of images.

The injection method of the present invention has advantages over current methods in that it is able to provide flow rates in excess of 6 cc/sec with a catheter that has a similar outer diameter to prior art catheters. The method allows for smaller caliber catheters to be used that would provide equal or greater flow rates than catheters of larger calibers. The benefits of smaller diameter catheters are well known. For instance, the amount of blood flow area in the vein where the catheter is placed can be increased, thereby reducing the incidence of thrombus formation, and providing less trauma to the patient.

As discussed earlier, the prior art injection catheter, illustrated in FIG. 1A, can place excessive stress on the walls between the lumens 24, 26 and the interior walls of the common tube portion 13. FIG. 2A illustrates the excessive stress being placed (see direction of arrows) on the internal wall and septum 66 between the lumens 24, 26 when the lumen 24 is being used for injection of fluid in a two-lumen catheter, while the same figure also shows the excessive stress being placed on the internal walls and septum 66 between the three lumens 24, 26, 27 of a three-lumen catheter.

By contrast, the present injection system reduces the pressure of the fluid into the catheter, while maintaining the flow rate and increasing the diameter, which results in a substantial reduction in pressure on the catheter's interior walls.

As illustrated in FIG. 2B, the present injection device injects the fluid through all lumens of the catheter simultaneously which results in equal pressure on the internal catheter walls and septum 66. This removes the tendency for the internal septum(s) 66 in the catheter, which over time may cause the catheter to distend and fail. This also allows for a thinner septum(s) 66 to be used, which provides more flow area and increases the catheter flow rate.

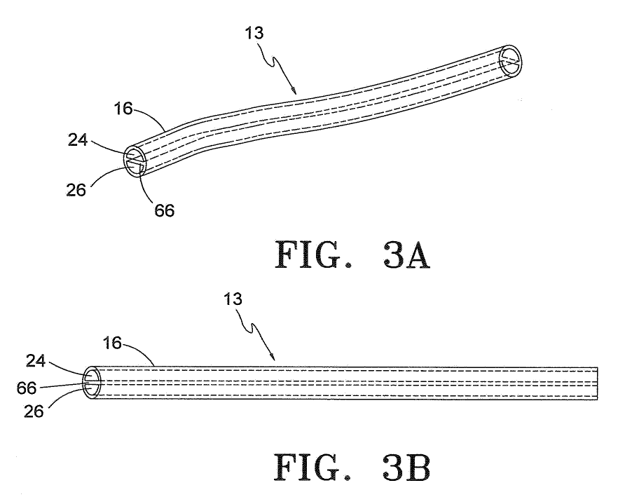

FIG. 3A depicts the movement of the common tube portion 13 when only one lumen 24 of the catheter is used for injection. Flow exits eccentrically resulting in an unequal thrust force at the distal end 16 and/or a whipping movement of the common tube portion 13.

FIG. 3B shows injection of a fluid through all catheter lumens 24, 26 simultaneously. Due to the equal thrust, movement of the distal end 16 remains stable.

An experiment was conducted to test the distal end 16 whip effect of one lumen of a dual lumen 5F catheter (see FIG. 1B) compared to a dual lumen 5F bifurcated catheter. A 64% glycerin/36% water mixture at body temperature was separately injected at a flow rate of 2.5 cc/sec into both the one lumen of the dual lumen catheter and the dual lumen catheter using an E-Z-EM EmpowerCT™ injector. The water/glycerin mixture was chosen in order to simulate the viscosity of Isovue®-370, a common contrast agent, which has a viscosity of 9.4 centipoise (cP) at body temperature. Data was recorded using a computer program called LabView™ (available from National Instruments of Austin, Tex.). In both the one lumen of the dual lumen catheter and the dual lumen catheter testing, the distal end 16 of the catheter 12 was unrestrained, and the injection pressure limit was set at 300 psi at the proximal end 22 of the catheter. This pressure value was chosen with the expectation that 300 psi is an upper limit of what injection pressures may reach during CT injections.

When the glycerin/water fluid mixture was injected into the one lumen of a dual lumen 5F catheter at a flow rate of 2.5 cc/sec, the distal end 16 of the catheter 12 started whipping immediately. As fluid began flowing through the catheter 12, the pressure, as detected at the proximal end of the catheter 22 adjusted to approximately 140 psi.

The glycerin/water mixture was then injected into the dual lumen 5F bifurcated catheter 12 at the same flow rate of 2.5 cc/sec and initial pressure of 300 psi. As fluid began to flow through the catheter 12, and the pressure reached about 140 psi, the distal end 16 of the dual lumen catheter 12 did not whip.

The 5F catheter used in the above-mentioned experiment has an outer diameter of approximately 0.066 inches, a septum 66 and wall thickness each of approximately 0.008 inches, a lumen width of approximately 0.020 inches, and a lumen height of approximately 0.046 inches. Although a 5F catheter was used in the above-mentioned experiment, a catheter of any size may be used in the method of the present invention, depending on the patient's needs and the desired treatment outcomes.

The internal diameter of the coupler device's common line 29 is approximately 0.072 inches. The outer diameter is approximately 0.142 inches. The bifurcate coupling element 47 has an internal diameter of approximately 0.138 inches. The extension lines 29, 40,42, 44 on each side of the bifurcate coupling element 47 may be between approximately 2 and 12 inches in length. The total length of the coupler device 28/58 may be between approximately 4.25 and 14.25 inches. The dimensions of the coupler device, bifurcate coupling element, and associated tubing are given herein for illustrative purposes only. These dimensions may be adjusted depending on the patient's need and the type of catheter used. However, the overall diameter of the bifurcate extension lines and coupler must be larger than the individual lumens of the catheter 12. Otherwise, the diameter of the coupler device 58/28 will limit the flow rate.

The above disclosure is intended to be illustrative and not exhaustive. Other modifications and changes in the details of the method illustrated herein can be made by those skilled in the art without departing from the spirit of the present invention. For example, multiple injectors may be used to deliver different fluids through a single catheter using the flow splitter or to vary pressures within the lumens to achieve specific flow patterns of the fluid or to direct the catheter tip in a desired location. Gas injected through one lumen may also be used to displace blood, allowing a liquid agent from another lumen to fill the space left in the blood vessel by the gas displacement. All connectors 6, 8, 9, 34, 36, 37 38 as shown in the drawings are Luer type connectors although other types of connectors may be used. Accordingly, the scope of the invention is not limited to the foregoing specification, but instead is given by the appended claims along with their full range of equivalents.

What is claimed is:

1. A fluid injection device comprising:
a multi-lumen catheter including:
a common tube having a set of lumens and a distal end adapted to be placed in a blood vessel; and
first and second extension tubes extending from a proximal end of the common tube with each extension tube having a lumen in communication with a respective one of the set of lumens; and
a coupler including:
an input lumen adapted to be connected to a fluid source; and
first and second output connectors respectively coupled to the first and second extension tubes, the coupler being adapted to receive the fluid through the input lumen and to output the received fluid simultaneously to the lumens of the first and second extension tubes.

2. The fluid injection device according to claim 1, wherein the coupler includes first and second extension lines in communication with the input lumen and respectively coupled to the first and second output connectors.

3. The fluid injection device according to claim 1, wherein the coupler includes a multi-lumen extension line having:
a common extension line containing the input lumen; and
first and second extension lines extending from the common extension line and respectively coupled to the first and second output connectors.

4. The fluid injection device according to claim 1, wherein each lumen of the set of lumens has a substantially semi-circular cross-section along the length of the common tube.

5. The fluid injection device according to claim 1, wherein the set of lumens include a third extension tube and the set of lumens are circumferentially uniformly spaced along the length of the common tube.

6. The fluid injection device according to claim 1, wherein the input lumen of the coupler is adapted to receive one or more of the following fluids: contrast media or dye, chemotherapeutic agents or drugs, anti-thrombotic agents, parenteral nutritional fluids, antibiotic or antiviral fluids, and IV fluids.

7. A method of injecting fluid through a multi-lumen catheter including a common tube having a set of lumens and first and second extension tubes extending from a proximal end of the common tube with each extension tube having a lumen in communication with a respective one of the set of lumens, the method comprising:
connecting the first and second extension tubes of the multi-lumen catheter to a source of the fluid;
simultaneously injecting the fluid into the lumens of the first and second extension tubes.

8. The method according to claim 7, wherein the step of connecting includes:
connecting a coupler to the first and second extension tubes of the multi-lumen catheter; and
connecting the coupler to the fluid source, wherein the coupler simultaneously output the fluid from the fluid source to the lumens of the first and second extension tubes.

9. The method according to claim 7, wherein the step of connecting includes:
connecting first and second extension lines of a second multi-lumen catheter to the first and second extension tubes of the multi-lumen catheter; and
connecting the second multi-lumen catheter to the fluid source, wherein the second multi-lumen catheter simultaneously outputs the fluid to the lumens of the first and second extension tubes of the multi-lumen catheter.

10. The method according to claim 7, wherein:
each lumen of the set of lumens has a substantially semi-circular cross-section along the length of the common tube; and
the step of injecting includes simultaneously injecting the fluid into the set of lumens having the substantially semi-circular cross-section.

11. The method according to claim 7, wherein:
the set of lumens include a third extension tube and the set of lumens are circumferentially uniformly spaced along the length of the common tube;
the step of injecting includes simultaneously injecting the fluid to the set of lumens that are circumferentially uniformly spaced.

12. The method according to claim 7, wherein the step of connecting the first and second extension tubes of the multi-lumen catheter includes connecting the extension tubes to a source of one or more of the following fluids: contrast media or dye, chemotherapeutic agents or drugs, anti-thrombotic agents, parenteral nutritional fluids, antibiotic or antiviral fluids, and IV fluids.
